(19) United States
(12) Patent Application Publication
(10) Pub. No.: US 2010/0064437 A1
(43) Pub. Date: Mar. 18, 2010

(54) MAT FOR MEDICAL IMAGE DIAGNOSIS APPARATUS AND BED FOR MEDICAL IMAGE DIAGNOSIS APPARATUS

(75) Inventors: Masahiro SUZUKI, Nerima-ku (JP); Kiyoyuki KODAMA, Chuo-ku (JP); Yoshiyuki MIYATANI, Tokyo (JP); Shinsuke TSUKAGOSHI, Nasushiobara-shi (JP); Tomokazu HARADA, Otawara-shi (JP)

Correspondence Address: OBLON, SPIVAK, MCCLELLAND MAIER & NEUSTADT, L.L.P., 1940 DUKE STREET, ALEXANDRIA, VA, 22314, US

(73) Assignee: TOSHIBA MEDICAL SYSTEMS CORPORATION, Otawara-shi (JP)

(21) Appl. No.: 12/547,127

(22) Filed: Aug. 25, 2009

(30) Foreign Application Priority Data
Sep. 17, 2008 (JP) 2008-237339
Jun. 15, 2009 (JP) 2009-142507

(51) Int. Cl. A61B 6/04

(52) U.S. Cl. 5/601

(57) ABSTRACT

A medical image diagnosis apparatus mat for laying a subject when imaging a medical image is configured so that a face on which the subject is laid can be switched between a state of a flat plate having a predetermined thickness and a state of a plate having the predetermined thickness and having a recessed part.

### BACKGROUND OF THE INVENTION

1. Field of the Invention

The present invention relates to a mat placed under a subject when the subject is laid for capturing an image and a bed with the mat attached, in a medical image diagnosis apparatus (an X-ray CT apparatus, an MRI apparatus, etc.) that generates a medical image.

2. Description of the Related Art

Conventionally, in an examination of the large intestine, observation with an endoscope directly inserted into the body is mainly performed. Moreover, another method of the examination is barium enema. In this examination method, barium and air are injected into the large intestine, and an X-ray image of a lesion site of the gastrointestinal mucosa is captured.

Furthermore, a technique of MSCT (Multi Slice CT (Computed Tomography)) is proposed in recent years. In this technique, a plurality of X-ray tomographic images are generated at a time in the rostrocaudal direction of a subject in an X-ray CT apparatus. Diffusion of MSCT has enabled a large intestine examination using an X-ray CT apparatus. In the large intestine examination by MSCT using an X-ray CT apparatus, X-ray tomographic images are generated by injecting air into the large intestine and inflating the large intestine, and a three-dimensional image is generated based on the X-ray tomographic images. Moreover, a technique that enables diagnosis by, for example, reconstructing original data based on the generated three-dimensional image and observing unevenness in the large intestine seen from the inside of the large intestine by a virtual endoscope is proposed (e.g., Japanese Unexamined Patent Publication No. 8-016813). Such a virtual endoscope enables observation of the large intestine, etc., from various angles. On the contrary, by an actual endoscope, it is not possible to observe polyp of the large intestine from a desired angle necessarily.

Therefore, accurate diagnosis may result from the diagnosis using the virtual endoscope.

Although an X-ray CT apparatus is used as an example in the above description, a medical image diagnosis apparatus such as an MRI (Magnetic Resonance Imaging) apparatus can also display an image by the virtual endoscope.

In order to perform the large intestine examination by MSCT using an X-ray CT apparatus without overlooking findings, it is necessary to move the residue remaining in the large intestine and display unevenness of the whole face of the wall of the large intestine.

Therefore, in the case of performing the large intestine examination by an X-ray CT apparatus, it is necessary to image a subject on his/her back and image the subject on his/her stomach.

However, in general, when the subject is on his/her stomach, the abdomen is pressed by the subject's own weight. Consequently, when the subject is imaged on his/her stomach, the abdomen is pressed and air escapes from the large intestine existing in the middle of the abdomen, which brings the abdomen into a collapsed state. To be specific, air cannot stay in the transverse colon. This is especially outstanding in the case of a fat subject. Therefore, the transverse colon, etc., is collapsed in the image, and the collapsed transverse colon, etc., cannot be displayed as a three-dimensional image. Consequently, some parts of the large intestine cannot be observed, and it becomes difficult to perform accurate diagnosis.

It commonly happens in other medical image diagnosis apparatuses such as an MRI apparatus that the abdomen is pressed and the large intestine is deformed when the subject is laid on his/her stomach, and it is difficult to generate a medical image in a state that the large intestine, etc., are not collapsed. Accordingly, it is also difficult to accurately diagnose in other medical medical image diagnosis apparatuses.

### SUMMARY OF THE INVENTION

The present invention was made in view of the circumstances, and an object of the present invention is to provide a medical image diagnosis apparatus mat and a medical image diagnosis apparatus bed, which reduce pressure against the abdomen when the subject is laid on his/her stomach.

A medical image diagnosis apparatus mat in a first aspect of the present invention is a medical image diagnosis apparatus mat for laying a subject when imaging a medical image, and a face on which the subject is laid can be switched between a state of a flat plate having a predetermined thickness and a state of a plate having the predetermined thickness and having a recessed part.

A medical image diagnosis apparatus bed in a second aspect of the present invention is a medical image diagnosis apparatus bed for laying a subject when capturing a medical image, and the bed includes: a top plate; a leg member configured to support the top plate; and a mat member placed on the top plate so that a subject is laid, The mat member is configured so that a face on which the subject is laid can be switched between a state of a flat plate having a predetermined thickness and a state of a plate having the predetermined thickness and having a recessed part.

The medical image diagnosis apparatus mat and the medical image diagnosis apparatus bed according to the present invention are configured to be switchable to a state of a plate in which part of the mat for laying the subject is recessed. Therefore, by placing the abdomen of the subject laid on his/her stomach into the recessed part, it is possible to reduce pressure against the abdomen by the subject's own weight, and it is possible to image without collapsing the large intestine, etc., even when the subject is laid on his/her stomach. Accordingly, the medical image diagnosis apparatus mat and the medical image diagnosis apparatus bed according to the present invention can contribute to effective diagnosis of the large intestine, etc., using a medical image diagnosis apparatus.

### BRIEF DESCRIPTION OF THE DRAWINGS

FIG. 1 is a perspective view of a medical image diagnosis apparatus bed including a medical image diagnosis apparatus mat according to a first embodiment.

FIG. 2 is a plan view of the medical image diagnosis apparatus mat according to the first embodiment.

FIG. 3A is a side view of the medical image diagnosis apparatus mat according to the first embodiment, on which a subject is laid on his/her back as a removable member is attached. FIG. 3B is a side view of the medical image diagnosis apparatus mat according to the first embodiment, on which a subject is laid on his/her stomach as the removable member is detached.

FIG. 4 is a perspective view of a medical image diagnosis apparatus bed including a medical image diagnosis apparatus mat according to a first modification.

FIG. 5A is a plan view of a medical image diagnosis apparatus mat according to a second modification. FIG. 5B is a plan view of a medical image diagnosis apparatus mat according to a third modification.

FIG. 6 is a perspective view of a medical image diagnosis apparatus bed including a medical image diagnosis apparatus mat according to a second embodiment.

FIG. 7 is a perspective view of a medical image diagnosis apparatus mat according to a third embodiment, which is mounted on a top plate, and from which a removable member is detached.

### DETAILED DESCRIPTION OF THE EMBODIMENTS

### First Embodiment

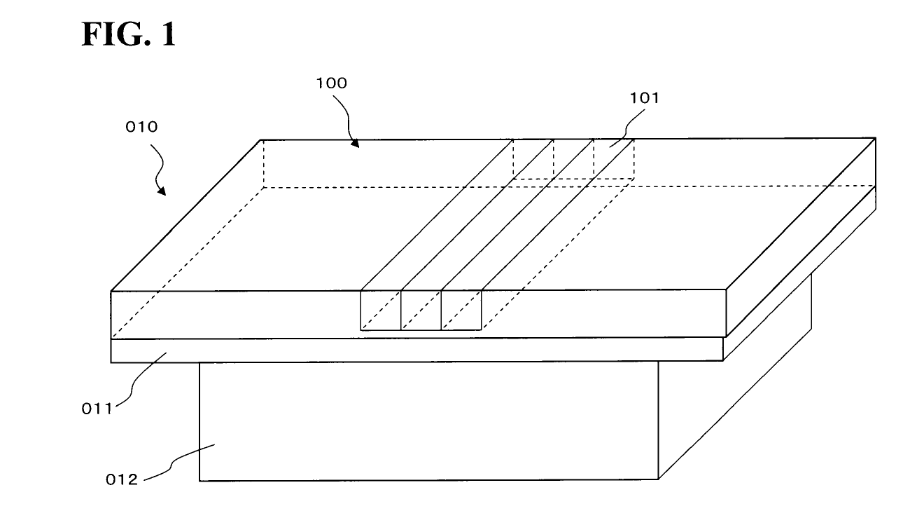

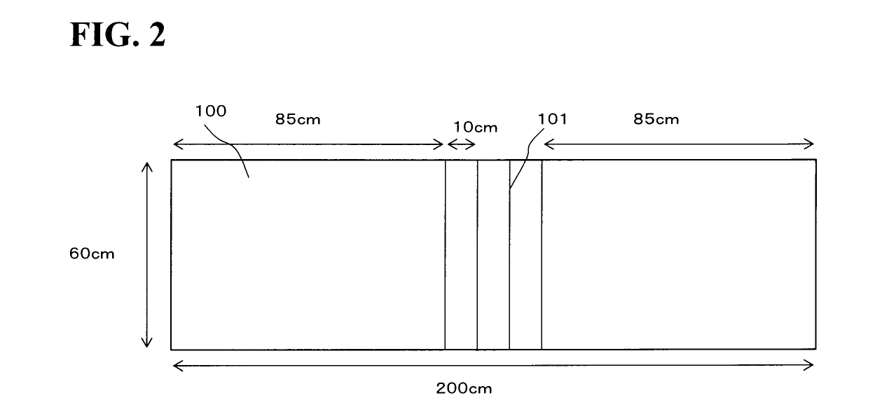

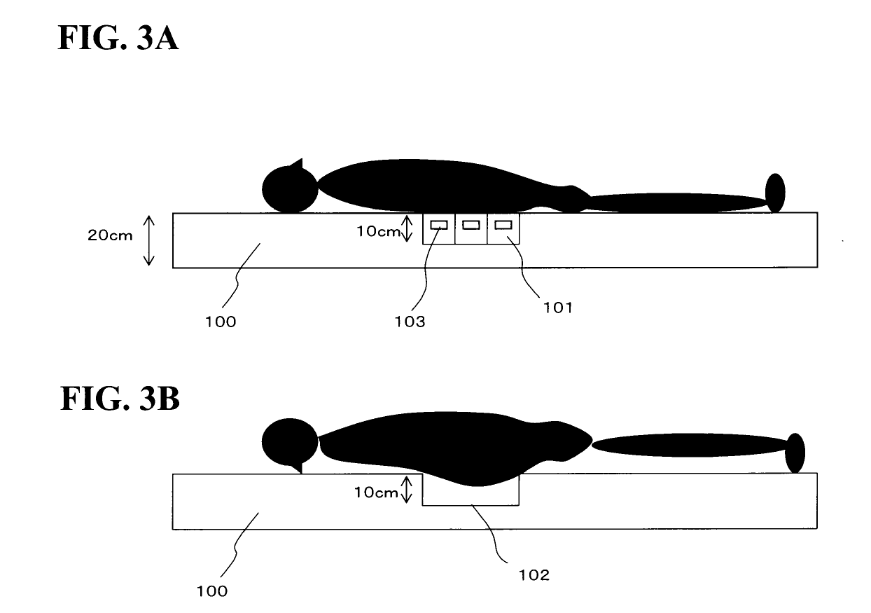

A medical image diagnosis apparatus mat according to a first embodiment of the present invention will be described below with reference to FIGS. 1 through 3. FIG. 1 is a perspective view in a state that the medical image diagnosis apparatus mat according to this embodiment is placed on a top plate. FIG. 2 is a plan view of the medical image diagnosis apparatus mat according to this embodiment. FIGS. 3A and 3B are side views of the medical image diagnosis apparatus mat according to this embodiment. FIG. 3A is a view showing a state that a subject is laid on his/her back on the medical image diagnosis apparatus mat in a flat plate state with a removable member fitted into a recessed part. FIG. 3B is a view showing a state that a subject is laid on his/her stomach on the medical image diagnosis apparatus mat in a partly recessed plate state with the removable member detached.

As shown in FIG. 1, a medical image diagnosis apparatus mat 100 (simply referred to as the “mat 100” hereinafter) according to this embodiment is placed on a top plate 011. In this embodiment, the top plate 011 is made of carbon. The top plate 011 is supported by a leg member 012 at a predetermined height from the floor. A bed 010 composed of the mat 100, the top plate 011 and the leg member 012 is equivalent to the medical image diagnosis apparatus bed of the present invention. In this case, the mat 100 is equivalent to the “mat member” in the present invention. In this embodiment, the mat 100 is fixed to the top plate 011 by MAGIC TAPE®. Although the mat 100 is fixed to the top plate 011 by MAGIC TAPE® in view of easiness to produce and detach in this embodiment, the mat 100 may be fixed by another method.

For example, the mat 100 may be fixed by hooks. Otherwise, the mat 100 may be fixed to the top plate 011 so as not to be detached.

As shown in FIG. 2, the mat 100 of this embodiment is 200 cm in longer length and 60 cm in shorter length. Moreover, as shown in FIG. 3, the mat 100 is 20 cm in thickness. The thickness of the mat 100 needs to be deep enough to form a recessed part 102 described later, and it is preferred to determine the thickness depending on the depth of the recessed part 102 to be formed. Furthermore, the mat 100 has a removable member 101 as shown in FIGS. 1, 2 and 3. The mat 100 is made of sponge and covered with polyvinyl chloride. The material of the mat 100 can be any material that does not affect image formation in a medical image diagnosis apparatus using the mat 100. To be specific, the material can be any material that does not appear in an X-ray image in a case that the mat is used in an X-ray CT apparatus, and can be any material that does not affect magnetism in a case that the mat is used in an MRI apparatus. For example, the material of the inside of the mat 100 may be expanded polystyrene foam. Although the removable member 101 is made of the same material as the other part of the mat 100 in this embodiment, the removable member 101 and the mat 100 may be made of different materials as far as the materials do not affect image formation in a medical image diagnosis apparatus to be used as described above. When the removable member 101 is detached, the mat 100 is brought into a state of a plate having a recessed part as shown in FIG. 3B. The recessed part of the mat 100 when all of the removable members 101 are detached as shown in FIG. 3B will be referred to as the recessed part 102 hereinafter. Moreover, a recess of the mat 100 formed by detaching part of the removable member 101 divided into a plurality of members from the mat 100 will be referred to as the “recess.”

The recessed part 102 is recessed approximately 30 cm in length in the longer direction of the mat 100, and is recessed over the entire length in the shorter direction of the mat 100. Moreover, the recessed part 102 is recessed 10 cm in depth as shown in FIG. 3A. In this embodiment, the recessed part 102 is placed in the middle in the longer direction of the mat 100. However, the recessed part 102 can be placed in a position of the abdomen of a subject when the subject is laid in a normal state on the mat 100. Since the subject is normally laid around the middle, the recessed part 102 can be placed around the middle of the mat 100. To be specific, “around the middle” refers to a range in which, when the abdomen of a subject is put on the recessed part 102, the feet or upper body of the subject does not protrude from the end in the longer direction of the mat 100 and the abdomen of the subject is away from the end in the shorter direction of the mat 100 so as not to protrude therefrom. However, since it is not so necessary to limit the length of the recessed part 102 in the shorter direction of the mat 100 as described later, the other portion of the recessed part 102 exceeds the abovementioned range as far as the minimum length of the recessed part 102 is within the abovementioned range.

The length of the recessed part 102 in the longer direction of the mat 100 can be equal to or more than a length enough to contain the abdomen of a person with a large abdomen (e.g., a fat person). On the other hand, it is meaningless if a portion other than containing the large intestine, etc., enters the recessed part 102 and the abdomen is strongly pressed in contact with the bottom of the recessed part 102. Therefore, the length needs to be equal to or less than a length such that the abdomen does not fall into the recessed part 102 to be strongly pressed in contact with the bottom. Since a range to scan the abdomen is generally 40 cm, the length of the recessed part 102 in the longer direction will be sufficient if it is 50 cm to allow a margin. A margin of 10 cm is given because there is a risk of affecting an image if there is a difference in level at the border of the scanning range. Empirically, a length of 20 cm or more is enough to contain the abdomen (for example, in the case of a person who is 180 cm tall, 20 cm is one sixth of the body height, and one sixth of a body height will be enough to contain the abdomen). Therefore, it is preferred that the length of the recessed part 102 in the longer direction of the mat 100 is 20 cm or more and 50 cm or less.

Further, the length of the recessed part 102 in the shorter direction of the mat 100 can be equal to or more than a length enough to contain the abdomen of a person with a large abdomen. Empirically, the abdomen can be contained into a recessed part of at least 20 cm (since the width of a person is approximately 35 cm at the maximum and 20 cm is four sevenths (nearly 60%) thereof, the recessed part will be enough to contain the abdomen). Therefore, it is preferred that the length of the recessed part 102 in the shorter direction of the mat 100 is 20 cm or more.

Furthermore, the depth of the recessed part 102 can be equal to or more than a depth enough to contain the abdomen of a person with a large abdomen. In a case that the material of the mat 100 is hard, it is expected empirically that the recessed part 102 can contain the abdomen if it is 1 cm or more. Therefore, the depth of the recessed part 102 needs to be 1 cm or more. In this regard, depending on the material of the mat 100, the body may sink and the abdomen may strongly come in contact with the bottom of the recessed part 102. Therefore, depending on the material of the mat 100, the depth of the recessed part 102 may be set to 5 cm or more. Thus, it is preferred that the depth of the recessed part 102 is 5 cm or more.

The mat 100 of the removable member 101 is composed of three members as shown in FIGS. 1 and 2. In this embodiment, the three members composing the removable member 101 have the same shapes.

The removable member 101 fits into the recessed part 102. When the removable member 101 is fitted into the recessed part 102, a portion excluding the recessed part 102 and removable member 101 of a face of the mat 100 to lay the subject forms a flat plane together with a face of the removable member 101 to lay the subject as shown in FIG. 3A, and the mat 100 becomes a flat plate. That is to say, the height of the removable member 101 and the depth of the recessed part 102 are substantially the same in length. In this embodiment, the height of the removable member 101 is 10 cm. The removable member 101 has substantially the same length as the shorter side of the mat 100 as shown in FIGS. 1 and 2, i.e., has a length of approximately 60 cm in this embodiment. Moreover, each of the three members composing the removable member 101 has a length of 10 cm in the shorter direction of the mat 100. Therefore, each of the three members composing the removable member 101 is a rectangular parallelepiped with sides 60 cm×10 cm×10 cm. Each of the three members composing the removable member 101 is made of sponge and is covered with polyvinyl chloride.

Besides, MAGIC TAPE® is disposed to the bottom of each of the three members composing the removable member 101, that is, to the face opposite to the face to lay the subject. MAGIC TAPE® is disposed to the recessed part 102 in a position corresponding to the MAGIC TAPE® of the removable member 101. The removable member 101 is fixed to the recessed part 102 by the MAGIC TAPE®. It may be fixed by another method. Otherwise, it is possible to use without fixing the removable member 101 to the recessed part 102.

The removable member 101 of this embodiment is provided with a handle 103 on the shorter side of the mat 100. Since the recessed part of this embodiment is recessed over the entire length in the shorter direction of the mat 100, it is possible to draw the removable member out of the recessed part 102 of the mat 100 by pulling the handle in the shorter direction of the mat 100. Consequently, it is possible to draw the removable member 101 to form the recessed part 102 without lifting the body of the subject, and it is possible to reduce the load on the subject.

Further, the three members composing the removable member can be separately detached. By detaching part of the three members composing the removable member 101, it is possible to change the length of the recess of the mat 100 in the longer direction of the mat 100.

For example, by drawing the two members on the right of the removable member 101 in FIG. 3A, it is possible to form a recess of 20 cm in the longer direction of the mat 100. Thus, it is possible to form a recess in accordance with the size of the abdomen of the subject. In this embodiment, the removable member 101 is composed of three members. However, by dividing the removable member 101 into more members, it is possible to compose the removable member 101 that makes it possible to form a recess more suitable for the size of the abdomen of the subject.

Further, although the shape of the bottom of the recessed part 102 is flat in this embodiment, it may be another shape as far as the abdomen of the subject can be contained. For example, the recessed part 102 may be configured so that the cross section of the bottom in the longer direction of the mat is curved like a mortar. In a case that the recessed part 102 is configured so that the bottom is curved, it is unnecessary to keep the abdomen not to come in contact with the bottom at all. The bottom moderately comes in contact with the abdomen, and is thereby capable of supporting the abdomen without deforming the intestine, etc.

### First Modification

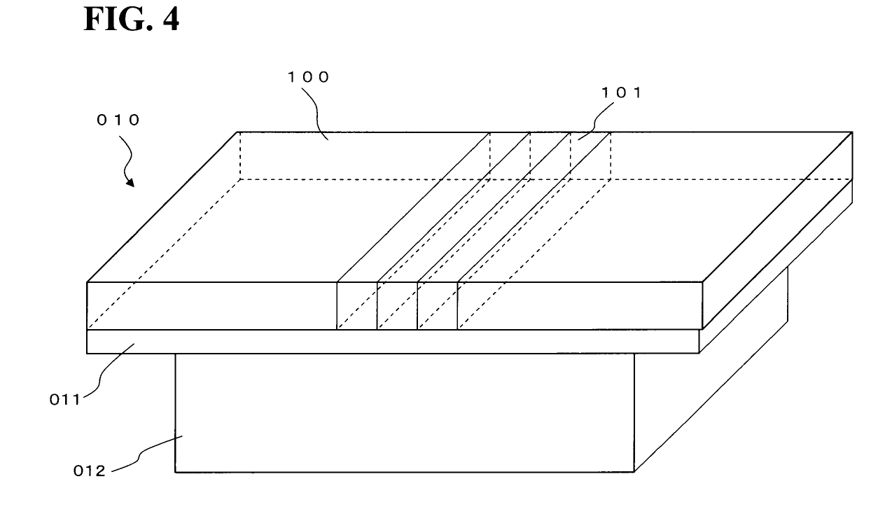

Next, a first modification of the medical image diagnosis apparatus mat according to this embodiment will be described. As shown in FIG. 4, the first modification has a configuration that the recessed part 102 passes through to the rear side (i.e., the opposite face to the face to lay the subject) of the mat 100. FIG. 4 is a perspective view of the medical image diagnosis apparatus mat and bed according to the first modification. The medical image diagnosis apparatus mat according to the aforementioned embodiment has a configuration that the recessed part 102 does not pass through as shown in FIG. 3B so that the mat 100 can be placed on any top plate 011 and is not displaced when the removable member 101 is detached. On the other hand, in a case that the mat 100 is formed integrally with the top plate 011, or in a case that the mat 100 is fixed to the top plate 011 by MAGIC TAPE® etc., the mat 100 will not be displaced even if the recessed part 102 passes through to the top plate 011 as shown in FIG. 4. Thus, the medical image diagnosis apparatus mat according to the first modification has a configuration that the recessed part 102 passes through the mat 100 and the mat 100 is separated. Consequently, the need for producing the mat 100 having a recess in view of the recessed part 102 is eliminated, and it becomes possible to facilitate the production of the mat 100.

### Second Modification

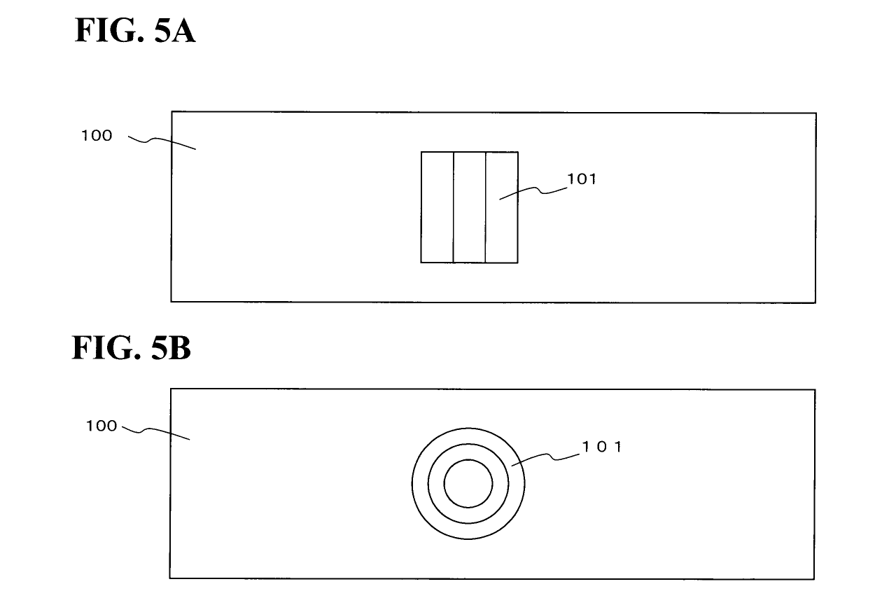

Next, a second modification of the medical image diagnosis apparatus mat according to this embodiment will be described. The second modification has a configuration that the recessed part 102 does not extend to the end in the shorter direction of the mat 100 as shown in FIG. 5A. FIG. 5A is a plan view of a medical image diagnosis apparatus mat according to the second modification. However, FIG. 5A shows a state that the removable member 101 is fitted into the recessed part 102, and does not plainly represent the recessed part 102. The medical image diagnosis apparatus mat according to the aforementioned embodiment has a configuration that the length in the shorter direction of the removable member 101 is the entire length of the mat 100 (i.e., from one end to the other end in the shorter direction of the mat 100). However, in general, the subject is laid substantially in the middle in the shorter direction of the mat 100 when imaged. Thus, the medical image diagnosis apparatus mat according to the second modification has a configuration that the removable member 101 is positioned at a predetermined distance away from each end in the shorter direction of the mat 100. Consequently, the removable member 101 is smaller in size and easier to detach, and it becomes possible to reduce the load on the operator removing the removable member 101.

### Third Modification

Next, a third modification of the medical image diagnosis apparatus mat according to this embodiment will be described. The third modification has a configuration that the shape of the recessed part 102 when viewed from above (i.e., from above on the side to lay the subject) is circular. However, FIG. 5B shows a state that the removable member 101 is fitted into the recessed part 102, and does not plainly represent the recessed part 102. FIG. 5B is a plan view of a medical image diagnosis apparatus mat according to the third modification. The medical image diagnosis apparatus mat according to the aforementioned embodiment and the medical image diagnosis apparatus mats of the first and second modifications have rectangular shapes (i.e., a square or a rectangle when viewed from above). On the other hand, the mat 100 according to the third modification has the recessed part 102 that is circular when viewed from above and further has the removable member 101 separated into a plurality of circular and columnar members (collectively referred to as the “ring-like members” hereinafter) that fit into the recessed part 102. The removable member 101 is configured so that the ring-like members can be separately detached. Consequently, it is possible to apply to subjects with different sizes of abdomens.

Moreover, the medical image diagnosis apparatus mat according to the third modification has a configuration that the recessed part 102 is circular and suitable for the abdomen, and it becomes possible to reduce the load on the subject to moderately support a portion other than the abdomen of the subject.

As described above, the medical image diagnosis apparatus mat according to this embodiment has a configuration that, when the subject is laid on his/her stomach, the abdomen is contained in a recessed part of the mat. Consequently, in the case of laying the subject on his/her stomach to capture a medical image of the abdomen, it is possible to generate a medical image in a state that the large intestine, etc., is not compressed and air reaches the whole large intestine, and it is possible to form a three-dimensional image of the large intestine that is not compressed. Thus, it becomes possible to effectively execute diagnosis using a virtual endoscopy image.

Further, although the aforementioned examples describe a configuration that it is possible to adjust the size of the recess in a direction to lay the subject on the bed 010 (i.e., a horizontal direction to the floor), the mat may be configured so that it is possible to adjust the depth of the recess (i.e., the size in the perpendicular direction to the floor).

As an example of this configuration, each of the members composing the removable member 101 shown in FIG. 1 is formed into a rectangular parallelepiped that is split into pieces at a certain depth, and is configured so that the depth of the recess can be adjusted by drawing a desired number of pieces of the removable member 101. As more pieces of the removable member 101 are drawn out, a deeper recess can be formed. Although the above configuration is described using the example of FIG. 1, it is also possible to produce a medical image diagnosis apparatus mat in which the size of a recess in the depth direction can be changed by using the removable member 101 split at a certain depth in other configurations (e.g., the configurations of the first through third modifications).

By this configuration, it becomes possible to adjust the size of a recess not only in the direction to lay the subject but also in the depth direction, and it becomes possible to more appropriately apply to subjects with different sizes of abdomens.

### Second Embodiment

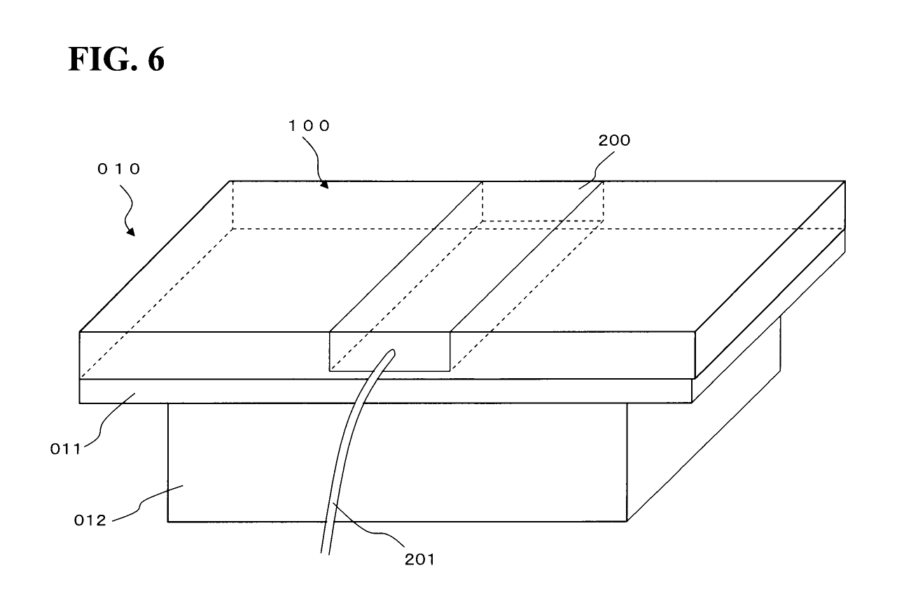

A medical image diagnosis apparatus mat according to a second embodiment of the present invention will be described below with reference to FIG. 6. FIG. 6 is a perspective view of a state that the medical image diagnosis apparatus mat according to this embodiment is placed on a top plate. The medical image diagnosis apparatus mat according to this embodiment is configured so that a recess is formed by deflating, though a recess is formed by detaching a removable member in the medical image diagnosis apparatus mat according to the first embodiment. Therefore, the configuration of the recess will be mainly described below. In the description below, members denoted by the same reference numerals as in FIG. 1 have the same configurations, except otherwise described.

As shown in FIG. 6, the medical image diagnosis apparatus mat 100 (referred to as the “mat 100” hereinafter) according to this embodiment has an abdomen placement part 200. To the abdomen placement part 200, an inlet/outlet tube 201 is connected. The size of the abdomen placement part 200 according to this embodiment is the same as the size of the recessed part 102 in the first embodiment.

The abdomen placement part 200 is hollow and is covered with polyvinyl chloride. The abdomen placement part 200 is hermetically sealed by polyvinyl chloride so as to prevent air from leaking outside except to the inlet/outlet tube 201. When air is injected, the abdomen placement part 200 inflates, and a surface of the abdomen placement part 200 in contact with the subject forms a flat plane together with a surface of the mat 100 other than the abdomen placement part 200 in contact with the subject. Consequently, the mat 100 is brought into a flat plate state. Moreover, the abdomen placement part 200 is recessed when air is discharged. Consequently, the mat 100 is brought into a partly recessed plate state. The abdomen placement part 200 in the deflated state is equivalent to the “recessed part” in the present invention.

Air filled in the mat 100 may be discharged by sucking out from outside, or may be discharged when the abdomen placement part 200 is pressed by the weight of the subject. In the configuration that air in the abdomen placement part 200 is discharged by the weight of the subject, a valve or the like is disposed so as to stop discharging when the pressure by the weight of the subject becomes lower than a predetermined pressure. Consequently, it becomes possible to support the abdomen of the subject at a moderate pressure, and it becomes possible to reduce the load on the subject.

The inlet/outlet tube 201 is connected to a pump or the like (not shown). Air conveyed from the pump is filled into the abdomen placement part 200 through the inlet/outlet tube 201. Moreover, the air filled in the abdomen placement part 200 is discharged outside through the inlet/outlet tube 201.

As described above, the medical image diagnosis apparatus mat according to this embodiment is configured so as to be brought into the flat plate state when air is injected into the abdomen placement part and brought into the partly recessed plate state when air is discharged from the abdomen placement part. Thus, since it is possible to switch the states by taking the air in and out, it is easy to switch the states and it is possible to switch the states without moving the subject, with the result that it is possible to reduce the load on the subject.

Furthermore, since it is possible to continuously change the size of the recessed part by the amount of the discharged or injected air, it is possible to provide the subjects with more proper sizes depending on the body types of the subjects.

Further, although the mat 100 is brought into the partly recessed state or the flat state by discharging or injecting air from or into the abdomen placement part 200 in this embodiment, it may have another configuration as far as air is injected so that a portion corresponding to the abdomen placement part 200 is brought into the recessed state.

For example, the mat 100 may have a configuration described below. A portion other than the abdomen placement part 200 of the mat 100 is hollow and is covered with polyvinyl chloride, and air can be injected into and discharged from the portion other than the abdomen placement part 200 of the mat 100. The abdomen placement part 200 has a fixed shape. When air is injected into the portion other than the abdomen placement part 200 of the mat 100, the height of the portion from the top plate 011 becomes more than the height of the abdomen placement part 200 from the top plate 011.

By thus configuring so that the portion other than the abdomen placement part 200 of the mat 100 becomes higher, it is possible to bring the portion of the abdomen placement part 200 of the mat 100 into the recessed state.

### Third Embodiment

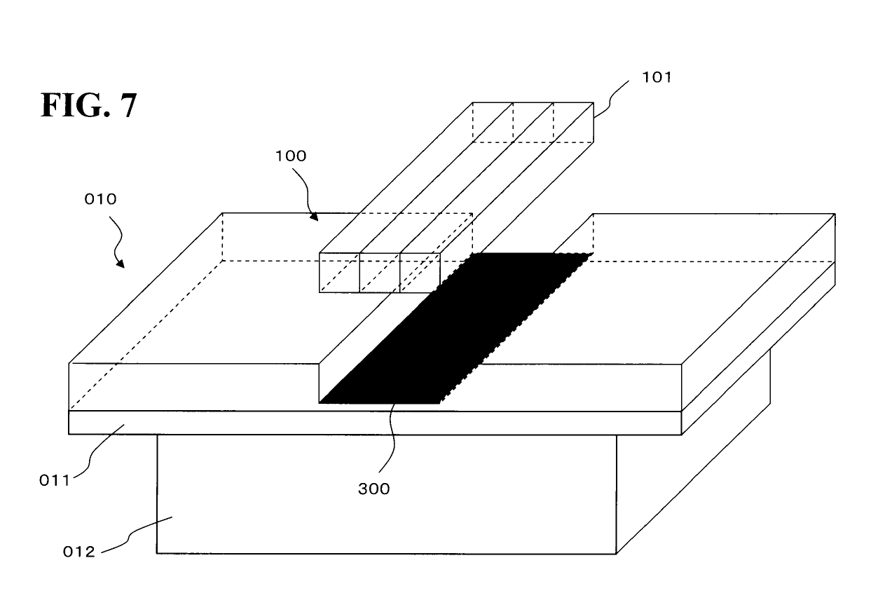

A medical image diagnosis apparatus mat according to a third embodiment of the present invention will be described below with reference to FIG. 7. FIG. 7 is a perspective view of a state that the medical image diagnosis apparatus mat according to this embodiment is placed on the top plate and the removable member is detached. The medical image diagnosis apparatus mat according to this embodiment has a configuration that, in the medical image diagnosis apparatus mat according to the first embodiment, the bottom of the recess of the face in contact with the removable member is made of a material having different roughness from the removable member. Therefore, the configuration of the recessed part will be mainly described below. In the following description, members denoted by the same reference numerals as in FIG. 1 have the same configurations, except otherwise described.

The mat 100 according to this embodiment includes the removable member 101 made of polyurethane.

Further, a bottom 300 (shown in gray in FIG. 7) of the recessed part when the removable member 101 of the mat 100 is detached is formed by a Teflon® sheet. A surface of a portion other than the bottom 300 of the mat 100 is made of polyurethane.

If surfaces made of the same material are joined, the surfaces may be meshed with each other and be hard to slide on each other.

Therefore, in a case that the whole surface of the mat 100 is made of polyurethane, a certain level of force may be necessary for drawing the removable member 101.

For this reason, in this embodiment, the bottom 300 is made of Teflon® as described above. Teflon® has a smoother surface than polyurethane. Therefore, the bottom 300 and the removable member 101 become hard to mesh with each other, and the smoothness can be increased. Consequently, in a case that the mat 100 is configured as in this embodiment, it becomes easier to draw the removable member 101 from the recessed part in the shorter direction of the bed 010.

Further, although Teflon® is used as the material of the bottom 300 in this embodiment, another material may be used as far as it can make the removable member 101 and the bottom 300 hard to mesh with each other. That is, any material can be used for the bottom 300 as far as it has different surface roughness from the removable member 101. For example, in a case that the removable member 101 is made of polyurethane, the bottom 300 may be made of a Nylon® sheet having a rougher surface than polyurethane.

Further, in this embodiment, only the portion of the bottom 300 of the recessed part is made of another material having different surface roughness. However, in order to make it easier to draw the removable member 101, the other faces of the recessed part having different surface roughness in contact with the removable member 101 may also be made of another material. Moreover, by differentiating the materials of the faces of the removable members 101 in contact with each other, surface roughness of the faces of the removable members 101 in contact with each other may be differentiated.

What is claimed is:

1. A medical image diagnosis apparatus mat for laying a subject when imaging a medical image, wherein a face on which the subject is laid can be switched between a state of a flat plate having a predetermined thickness and a state of a plate having the predetermined thickness and having a recessed part.

2. The medical image diagnosis apparatus mat according to claim 1, comprising a removable member configured to have almost the same thickness as the predetermined thickness to fit into the recessed part and to be detachable, wherein the face changes into the state of the plate having the recessed part by detaching the removable member.

3. The medical image diagnosis apparatus mat according to claim 2, wherein the removable member is divided into a plurality of members split in a longer direction of the mat, and the plurality of members can be individually detached.

4. The medical image diagnosis apparatus mat according to claim 2, wherein the removable member has a substantially rectangular parallelepiped shape and the removable member is configured to be detachable by horizontally moving in a shorter direction of the mat.

5. The medical image diagnosis apparatus mat according to claim 4, wherein at least a bottom of the recessed part of faces of the recessed part in contact with the removable member is made of a material having different roughness from a material of a surface of the removable member.

6. The medical image diagnosis apparatus mat according to claim 4, wherein a length in the shorter direction of the removable member is a different length from a length in the shorter direction of a portion other than the removable member of the mat.

7. The medical image diagnosis apparatus mat according to claim 2, wherein the recessed part has a circular shape when viewed from above and the removable member fitting into the recessed part has a substantially columnar shape.

8. The medical image diagnosis apparatus mat according to claim 7, wherein the removable member is divided into a plurality of concentric ring-like members and the plurality of members can be individually detached.

9. The medical image diagnosis apparatus mat according to claim 1, wherein the part is sectioned from the other portion of the mat to be switched from the state of the flat plate to the state of the plate having the recessed part when air in the part is discharged, and switched from the state of the plate having the recessed part to the state of the flat plate when air is injected into the recessed part.

10. The medical image diagnosis apparatus mat according to claim 1, wherein the recessed part is positioned in middle in a longer direction of the mat.

11. The medical image diagnosis apparatus mat according to claim 1, wherein a length of the recessed part in a longer direction of the mat is equal to or more than 20 cm and equal to or less than 50 cm.

12. The medical image diagnosis apparatus mat according to claim 1, wherein a depth of the recessed part is 1 cm or more.

13. The medical image diagnosis apparatus mat according to claim 1, wherein a length of the recessed part in a shorter direction of the mat is 20 cm or more.

14. A medical image diagnosis apparatus bed for laying a subject when capturing a medical image, the bed comprising:
a top plate;
a leg member configured to support the top plate; and
a mat member placed on the top plate so that a subject is laid, wherein the mat member is configured so that a face on which the subject is laid can be switched between a state of a flat plate having a predetermined thickness and a state of a plate having the predetermined thickness and having a recessed part.
